(19) United States
(12) Patent Application Publication
(10) Pub. No.: US 2008/0105263 A1
(43) Pub. Date: May 8, 2008

(54) Esophageal intubation and airway management system and method

(76) Inventor: Kishor B. Jadhav, Topeka, KS (US)

Correspondence Address: MARK BROWN, 4700 BELLEVIEW SUITE 210, KANSAS CITY, MO, 64112, US

(21) Appl. No.: 11/593,926

(22) Filed: Nov. 7, 2006

(51) Int. Cl. A61M 16/04; A61B 1/267; A61B 1/01

(52) U.S. Cl. 128/207.15; 128/207.14; 128/204.22; 600/114

(57) ABSTRACT

An esophageal intubation system includes a main tube with a tapered body, proximal and distal ends and a bore extending between and open at the ends. The main tube is inserted into the oropharynx and its distal end extends into the upper esophagus in order to provide a conduit for esophagoscopes, gastroscopes and related diagnostic and treatment equipment. A first auxiliary tube assembly delivers O₂ close to the larynx. A second ancillary tube assembly samples CO₂ from the larynx region. An esophageal intubation method includes the steps of moderately sedating a patient, placing a main esophageal tube in the oropharynx and partway into the upper esophagus, passing an esophagoscope or a gastroscope through the main tube and into the esophagus or stomach while minimizing the gag reflex, delivering O₂ and sampling CO₂ from the pharynx region.

### BACKGROUND OF THE INVENTION

1. Field of the Invention

The present invention relates generally to airway management in connection with endoscopic procedures, and in particular to an endoscope esophageal tube with oxygen supply and carbon dioxide sensing ports.

2. Description of the Related Art

Esophagoscopes comprising fiber optics in flexible tubes and gastroscopes are introduced into the esophagus and stomach for diagnostic procedures under moderate or deep sedation in spontaneously breathing patients. However, such procedures present a number of concerns and potential problems. For example, a strong gag reflex is commonly triggered by the passage of instrumentation through the oropharynx (oral cavity). Aspiration of gastric contents into the larynx is another significant concern. Yet another concern relates to maintaining adequate oxygenation during moderate to deep sedation while the procedure is being performed.

Although some of the aforementioned concerns can be addressed by placing the patient under general anesthesia, it is generally preferable for the patient to be alert with only moderate, if any, sedation during diagnostic procedures of the esophagus and stomach. For example, deep sedation requires more oxygenation to maintain normal oxygen levels in the blood than is required with moderate sedation of a spontaneously breathing patient.

Previous esophagus and stomach diagnostic procedures used nasal cannula for administering O₂ to moderately sedated patients. However, nasal cannula typically only provided a maximum O₂ concentration of about 40%.

Another area of concern involving previous procedures and equipment relates to the required skills for administering them. Effectively placing esophagoscopes and gastroscopes without gagging the patient typically requires a relatively high level of skill and experience. Trainees and other practitioners with relatively little experience tend to elicit more gag reflexes and hypoxia in their patients. Consequently, deeper sedation may be indicated in order to suppress the gag reflex more commonly encountered by inexperienced medical practitioners, even though deep sedation involves attendant risks and disadvantages and is therefore to be avoided if possible.

Other types of equipment adapted for esophageal/tracheal placement include esophageal stethoscopes, temperature probes, gastric tubes and Doppler instruments for measuring cardiac output during cardiac surgery.

Heretofore there has not been available a system and method for esophageal intubation, O₂ delivery and CO₂ monitoring with the advantages and features of the present invention, including greater safety, comfort and tolerability for patients undergoing endoscopic procedures under moderate to deep sedation, and greater technical ease for medical practitioners administering such procedures.

### SUMMARY OF THE INVENTION

In the practice of an aspect of the present invention, a system is provided for esophageal intubation, which includes a tapering esophageal tube or sheath, an O₂ delivery port and a CO₂ sampling port. O₂ is delivered relatively close to the larynx and in relatively high concentrations, i.e. up to approximately 90%. CO₂ can be monitored in order to indicate breathing rate and degree of sedation, simultaneously with the implementation of an esophageal diagnostic procedure and O₂ delivery. The esophageal tube prevents aspiration of gastric contents coming into the oral cavity and larynx, because the esophageal tube bypasses the larynx. The esophageal tube also serves as a conduit to pass the esophagoscope or gastroscope, and prevents a strong gag reflex initiated by such instrumentation.

### BRIEF DESCRIPTION OF THE DRAWINGS

FIG. 1 is a perspective view of an esophageal intubation system embodying an aspect of the invention.

FIG. 2 is a side elevational view of the intubation system in a patient.

FIG. 3 is in enlarged, cross-sectional view thereof taken generally along line 3-3 in FIG. 1.

FIG. 4 is a front elevational view thereof.

FIG. 5 is a perspective view of an esophageal intubation system embodying a first modified aspect of the present invention, including an optional inflatable cuff.

FIG. 6 is a perspective view of an esophageal intubation system embodying a second modified aspect of the present invention, including an optional securing strap.

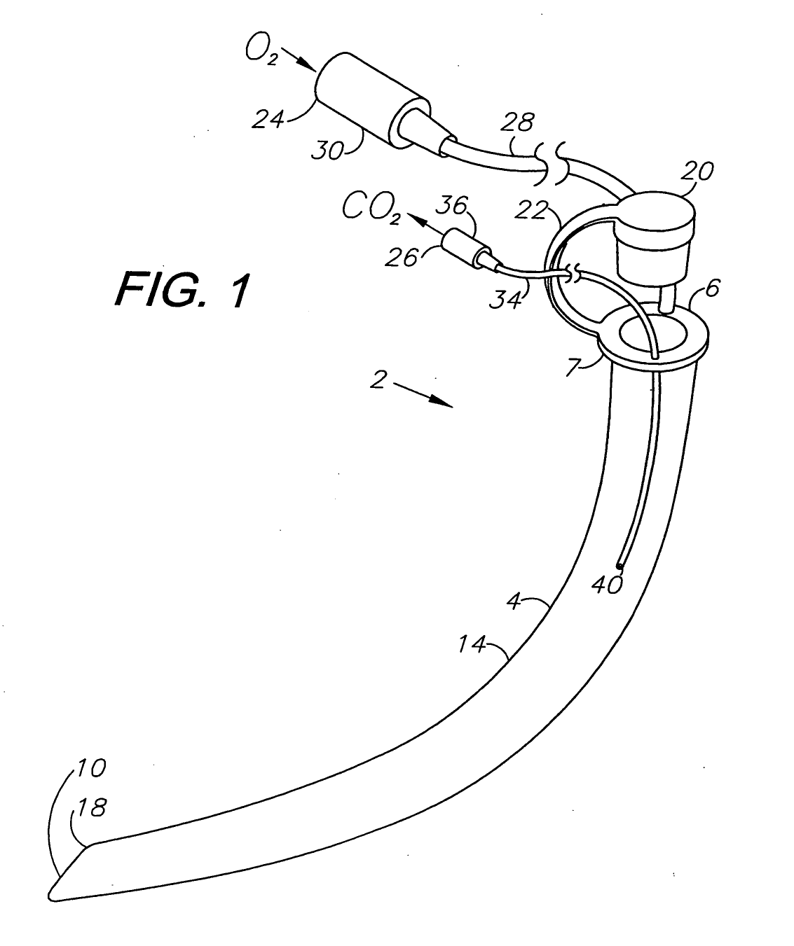

### DETAILED DESCRIPTION OF THE PREFERRED EMBODIMENTS

### I. Introduction and Environment

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Certain terminology will be used in the following description for convenience in reference only and will not be limiting. For example, up, down, front, back, right and left refer to the invention as oriented in the view being referred to. The words “inwardly” and “outwardly” refer to directions toward and away from, respectively, the geometric center of the embodiment being described and designated parts thereof. Forwardly and rearwardly are generally in reference to the direction of travel, if appropriate. Said terminology will include the words specifically mentioned, derivatives thereof and words of similar meaning.

Referring to the drawings in more detail, the reference numeral 2 generally designates an esophageal intubation system embodying an aspect of the present invention. The intubation system 2 includes an esophageal tube 4 with a proximal end 6 including a flange 7 adapted for placement external to a patient 8, a distal end 10 adapted for placement within the upper end of the esophagus 12 of the patient 8, a tube body 14 and a tube bore 16 extending between and open at the tube ends 6, 10. The esophageal tube 4 preferably comprises a relatively soft, pliable plastic material and functions as a conduit for placement of and esophagoscope or a gastroscope. The tube body 14 and bore 16 have distally-converging tapered configurations whereby the proximate end 6 has greater diameters, e.g. about 0.75 and 0.625 inches (OD and ID respectively) than the distal end, with a diameters of about 0.625 and 0.5 (OD and ID respectively). The distal end 10 preferably has an angular or beveled configuration with respect to the body 14 and smooth radiused margins 18 for patient comfort.

A proximal plug 20 is provided for insertion in the bore 16 at the proximal end 6 for selectively closing same and can be tethered to the body 14 by a suitable flexible tether 22. For example, closing the main tube proximal end 6 may be indicated in order to protect the esophagus 12 when procedures are not being performed and while providing breathing assistance, for example with a bag and mask, for depressed breathing due to deep sedation. First and second auxiliary tube assemblies 24, 26 are provided along a proximal section of the tube 4 adjacent to the proximal end 6. The first auxiliary tube assembly 24 includes a tubular extension section 28, which extends proximally of the main tube 4 proximal end 6 and is adapted for connection to a O₂ source, such as an oxygen tank via a connector, such as a Leur lock 30. The first auxiliary tube assembly 24 terminates distally at a first auxiliary tube port 32, which is open at the outside of the main tube body 14, which can be located about four inches from the proximal end 6. The second auxiliary tube assembly 26 likewise includes a tubular extension section 34 terminating at a Leur lock connector 36, which can be connected to an external sensor, such as a CO₂ sensor for monitoring the patient's breathing and sedation. The second auxiliary tube assembly 26, like the first auxiliary tube assembly 24, extends along a proximal section of the main tube body 14 adjacent to its proximal end 6 and terminates at a second auxiliary tube port 40, which is open at the exterior of the main tube body 14 and is adapted for CO₂ intake and can also be located about four inches from the proximal end 6.

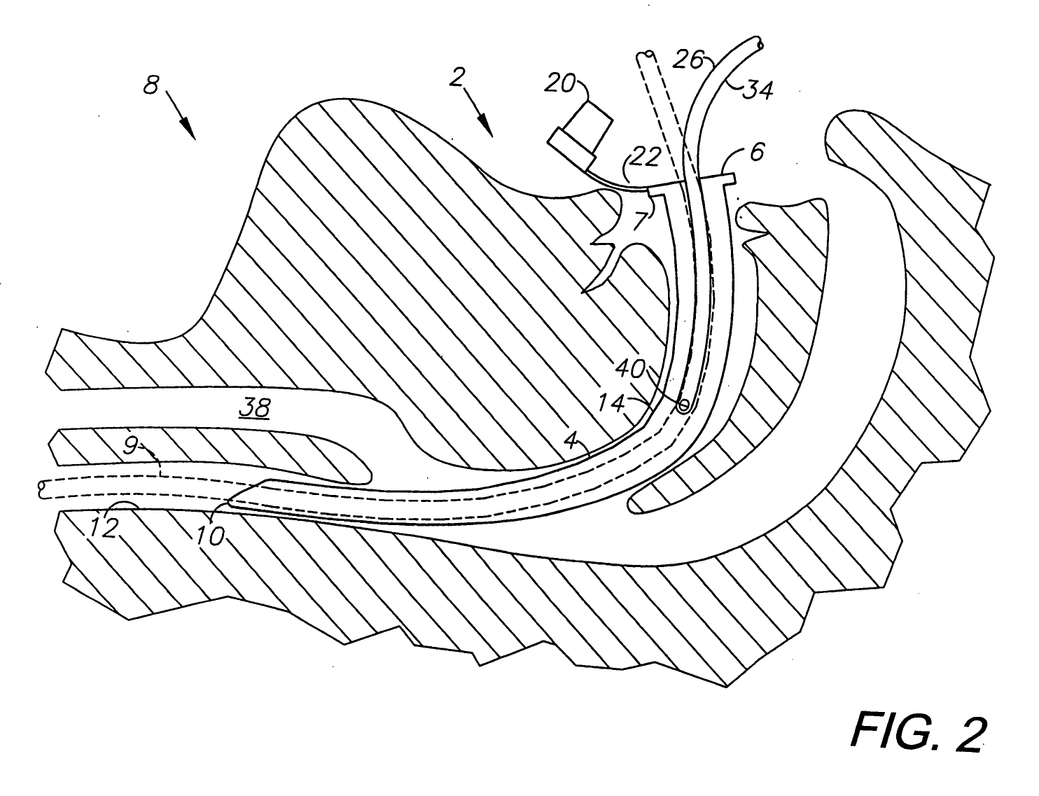

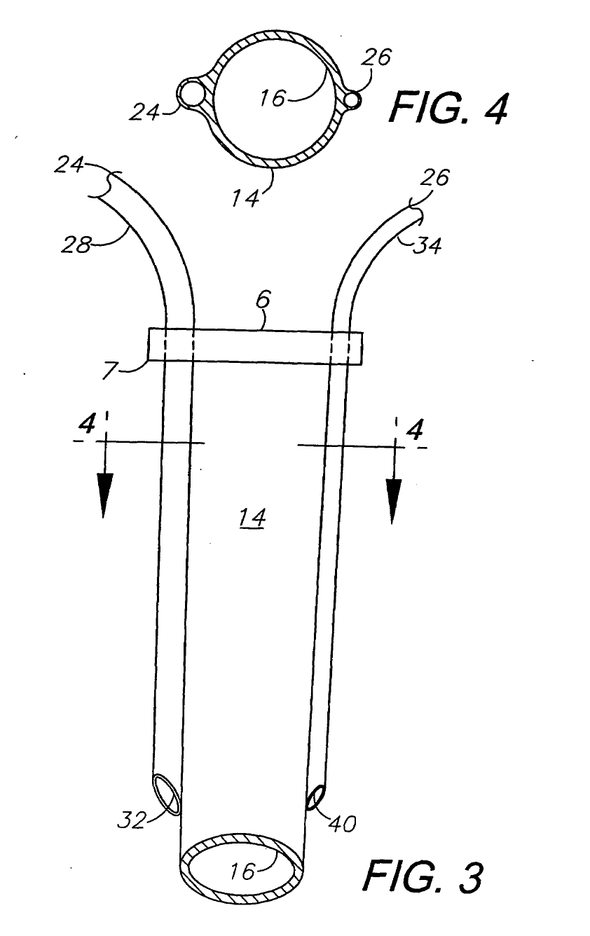

In operation, the esophageal intubation system 2 is adapted for placement orally (as shown in FIG. 2) whereby the distal end 10 extends slightly into the upper end of the esophagus in order to provide a sheath for insertion of tubular equipment such as endoscopes, esophageal stethoscopes, temperature probes, nasal gastric (NG) tubes. The general location of such equipment is shown in broken lines in FIG. 2 and designated by the reference numeral 9. The gag reflex is effectively suppressed because the tube body 14 functions as a protective sheath preventing direct contact between the patient and such equipment in the oral, nasal and upper esophageal cavities and passages. The tube proximal end 6 can be closed with the closure plug 20 while breathing is assisted, e.g. with bag and mask administering O₂. Moreover, the esophageal tube 14 protects against aspiration by providing a discharge path for the stomach contents while maintaining a clear passage for O₂ through the first auxiliary tube assembly 24. Relatively high concentrations of O₂ (e.g. approximately 90%) can be delivered in close proximity to the larynx. Breathing rate and sedation can be monitored via the second auxiliary tube assembly 26. Esophageal and stomach endoscopic and related diagnostic and treatment procedures can be more easily performed with the system 2, which tends to avoid the gag reflex during manipulation and placement of endoscopes and other equipment. Such procedures can thereby be performed by inexperienced medical practitioners with relatively little training and experience, including trainees. The airway (trachea) 38 is protected from aspiration of the gastric contents during the procedure. Without limitation on the usefulness of the present invention in a wide variety of procedures with patients under general anesthesia, the esophageal intubation system 2 can provide a conduit for many tubes passing through the esophagus, such as: esophageal stethoscopes; esophageal temperature probes; instrumentation for esophageal echocardiography for use during cardiac surgery; gastric tubes; and other equipment and instrumentation.

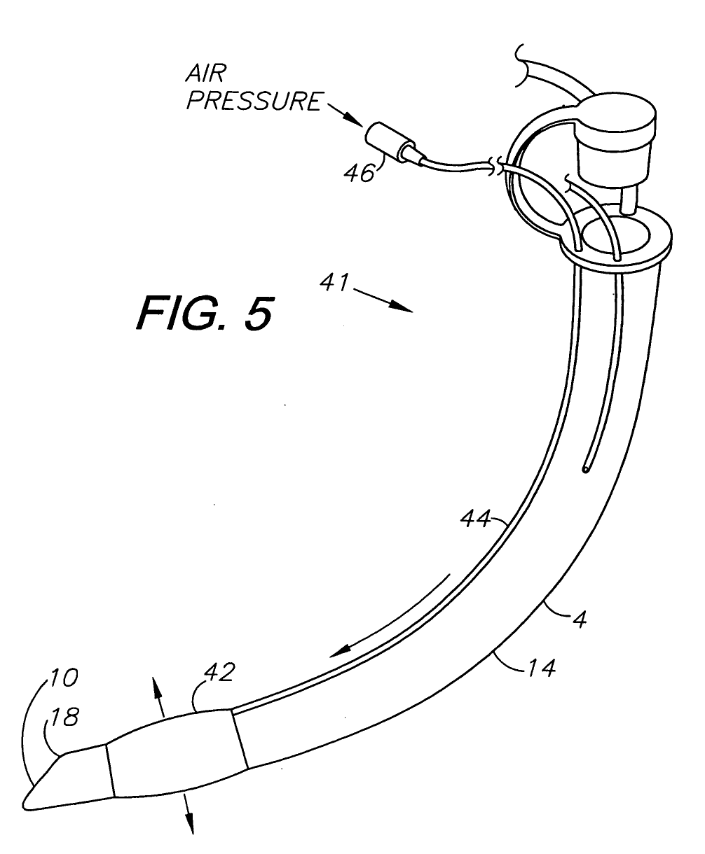

FIG. 5 shows an esophageal intubation system 41 embodying a first modified aspect of the present invention including an optional inflatable cuff 42. The cuff 42 is inflatable via a conduit 44 extending along the tube body 14 and terminating at a suitable connector 46, which can be connected to an air syringe. The connector 46 can be opened for deflating the cuff 42. Inflating the cuff 42 may be indicated in order to affect a more complete sealing closure of the esophagus if needed.

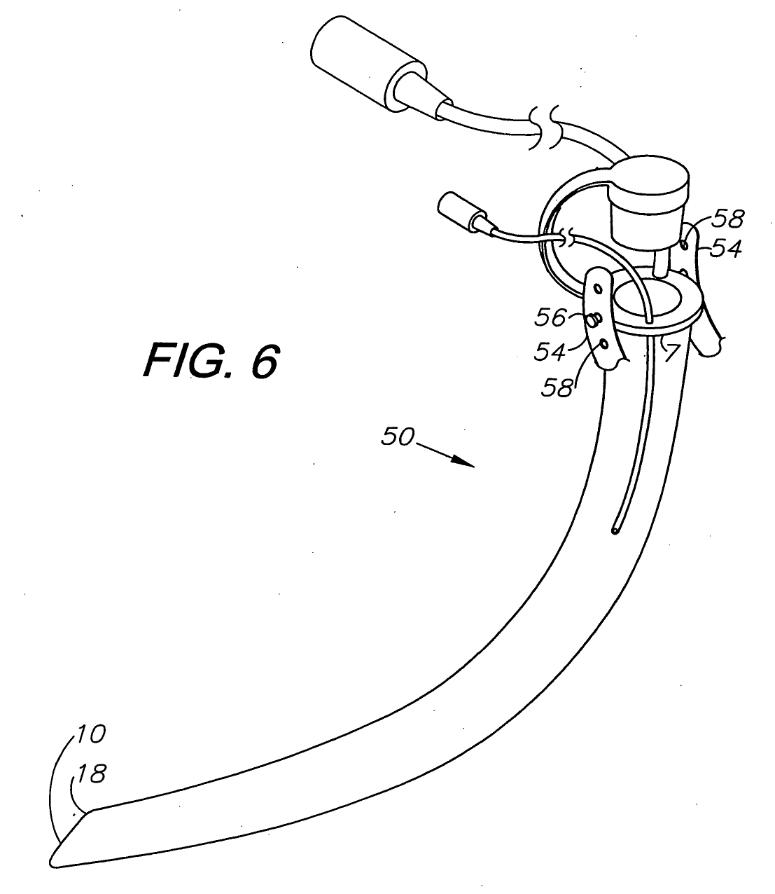

FIG. 6 shows an esophageal intubation system 50 embodying a second modified aspect of the present invention including an optional securing strap 54, which can be adjustably and releasably attached to the tube proximal end by placing anchors 56 extending from the flange 7 in strap receivers 58. The strap 54 is adapted for encircling the head or neck of the patient and retaining the tube system 50 securely in place.

It is to be understood that the invention can be embodied in various forms, and is not to be limited to the examples discussed above. Other components and configurations can be utilized in the practice of the present invention.

Having thus described the invention, what is claimed as new and desired to be secured by Letters Patent is:

1. An esophageal intubation system, which comprises:
a main esophageal tube with proximal and distal ends, a tube body extending between the ends and a tube bore extending through the body and open at the ends; and
said tube having a flared configuration adapted for facilitating placement of said distal end in the upper end of the esophagus.

2. The intubation system according to claim 1, which includes:
an auxiliary tube assembly including a coextensive portion extending along said main tube in proximity to its proximal end and a port open at said main tube body.

3. The intubation system according to claim 1, which includes:
said auxiliary tube assembly comprising a first auxiliary tube assembly; and
a second auxiliary tube assembly including a coextensive portion extending along said main tube in proximity to its proximal end and a port open at said main tube body.

4. The intubation system according to claim 3 wherein each said auxiliary tube assembly includes an extension extending beyond said body proximal end and terminating at a connector adapted for supplying O₂ and/or receiving CO₂ from a patient.

5. The intubation system according to claim 3 wherein said auxiliary tube ports are located in a patient's oral cavity with the system placed in the patient.

6. The intubation system according to claim 1 wherein:
said main esophageal tube has a tapered configuration with said proximal end having a larger diameter and said distal end having a smaller diameter; and
said main esophageal tube distal end has an angular configuration.

7. The intubation system according to claim 3 wherein:
said main esophageal tube proximal end includes a radially-extending flange; and
said auxiliary tube assemblies extend through said flange.

8. The intubation system according to claim 1, which includes:
an inflatable cuff encircling said main esophageal tube in proximity to its distal end; and
a cuff conduit connected to said cuff and extending along said main esophageal tube to said proximal end thereof, said cuff conduit being adapted for communicating air to said cuff for inflating said cuff.

9. The invention according to claim 1 wherein said main esophageal tube configuration is pre-curved and corresponds to the relative positions of the oropharynx, the larynx and the esophagus.

10. The invention according to claim 1, which includes:
a main esophageal tube plug with a closed position in said proximal end; and
a plug strap connecting said plug to said main tube proximal end.

11. The invention according to claim 7, which includes:
a pair of strap anchors extending radially outwardly from said flange; and
an elastic strap with multiple apertures each adapted to receive a respective strap anchor.

12. An esophageal intubation system, which comprises:
a main esophageal tube with proximal and distal ends, a tube body extending between the ends and a tube bore extending through the body and open at the ends;
said main esophageal tube having a configuration adapted for facilitating placement of said distal end in the upper end of the esophagus, said tube configuration comprising a tapered configuration with said proximal end having a larger diameter and said distal end having a smaller-diameter, angular configuration;
said main esophageal tube configuration being pre-curved and corresponding to the relative positions of the oropharynx, the larynx and the esophagus;
said main esophageal tube having a radially-extending flange at its proximal end;
a first, O₂ delivery auxiliary tube assembly including a coextensive section extending along said main tube in proximity to its proximal end, a port open at said main tube body, an extension section extending from said flange and a connector adapted for connection to an oxygen source;
a second, CO₂ sampling auxiliary tube assembly including a coextensive section extending along said main tube in proximity to its proximal end, a port open at said main tube body, an extension section extending from said flange and a connector adapted for connection to a CO₂ sampling device;
a main esophageal tube plug with a closed position in said proximal end;
a plug strap connecting said plug to said main tube proximal end;
an inflatable cuff encircling said main esophageal tube in proximity to its distal end; and
a cuff conduit connected to said cuff and extending along said main esophageal to said proximal end thereof, said cuff conduit being adapted passing air to said cuff for inflating said cuff.

13. A patient intubation and airway management method, which comprises the steps of:
mildly sedating the patient;
inserting an esophageal tube system in the patient's oropharynx and partly into the patient's upper esophagus;
providing the tube system with an O₂ delivery auxiliary tube assembly including a coextensive portion extending along said main tube in proximity to its proximal end and a port open at said main tube body;
connecting the O₂ delivery auxiliary tube assembly to an O₂ source;
delivering O₂ through the O₂ delivery auxiliary tube assembly in the larynx region;
providing the tube system with a CO₂ sampling auxiliary tube assembly including a coextensive portion extending along said main two in proximity to its proximal end and a port open at said main tube body;
connecting the CO₂ sampling tube assembly to CO₂ sampling equipment;
obtaining CO₂ samples from the larynx region and sampling same;
passing an esophagoscope or a gastroscope through said main tube passage into the esophagus and/or the stomach;
performing a diagnostic or treatment procedure in the esophagus and/or the stomach; and
minimizing a gag reflex reaction to said equipment.

14. The method according to claim 13, which includes the additional step of:
providing each said auxiliary tube assembly with an extension extending beyond said body proximal end and terminating at a connector adapted for supplying O₂ and/or receiving CO₂ from a patient.

15. The method according to claim 13, which includes the additional steps of:
providing said main esophageal tube proximal end with a radially-extending flange; and
extending said auxiliary tube assemblies through said flange.

16. The method according to claim 13, which includes the additional steps of:
providing said main esophageal tube with an inflatable cuff encircling said main esophageal tube in proximity to its distal end and a cuff conduit connected to said cuff and extending along said main esophageal tube to said proximal end thereof; and communicating pressurized air through said cuff conduit for inflating said cuff in the esophagus.

17. The method according to claim 13, which includes the additional steps of:
providing a main esophageal tube plug with a closed position in said proximal end; and
providing a plug strap connecting said plug to said main tube proximal end.

18. The method according to claim 13, which includes the additional steps of:
providing said main esophageal tube with a pre-curved configuration corresponding to the relative positions of the oropharynx, the larynx and the esophagus.

19. The method according to claim 13, which includes the additional steps of:
providing a pair of strap anchors extending radially outwardly from said flange;
providing an elastic strap with multiple apertures each adapted to receive a respective strap anchor; and
receiving said strap anchors in said apertures and securing said the esophageal intubation system by partly encircling the patient's head or neck with said strap.
